(19) United States
(12) Patent Application Publication
(10) Pub. No.: US 2026/0200841 A1
(43) Pub. Date: Jul. 16, 2026

(54) PROCESS FOR THE PREPARATION OF 2-ALKYLSULFONYL SUBSTITUTED BENZOIC ACID DERIVATIVES

(71) Applicant: ADAMA AGAN LTD., Ashdod (IL)

(72) Inventors: Michael GRABARNICK, Metar (IL); Shaoxiang WU, Haidian District, Beijing (CN); Rui WANG, Haidian District, Beijing (CN); Jing LI, Haidian District, Beijing (CN)

(73) Assignee: ADAMA AGAN LTD., Ashdod (IL)

(21) Appl. No.: 19/134,526

(22) PCT Filed: Nov. 30, 2023
(86) PCT No.: PCT/IL2023/051228

§ 371 Date: May 30, 2025

(30) Foreign Application Priority Data
Dec. 1, 2022 (WO) PCT/CN2022/135836

(51) Int. Cl. C07C 315/02; C07C 315/06; C07D 231/20

(52) U.S. Cl. CPC C07C 315/02; C07C 315/06; C07D 231/20

(57) ABSTRACT

The invention provides a method for preparing 2-alkylsulfonyl substituted benzoic acid derivatives of formula (I) by conversion of 2-alkylthio substituted benzonitrile compound of formula (II), which are useful as intermediates in the preparation of herbicidally active compounds like pyrasulfotole or isoxaflutole.

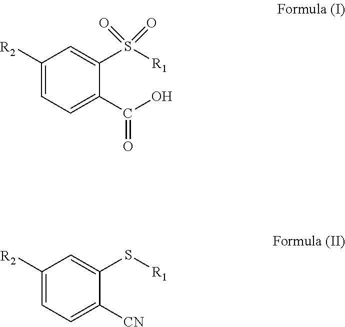

### FIELD OF THE INVENTION

This invention relates to a process for preparing 2-alkylsulfonyl substituted benzoic acid compound of formula (I),

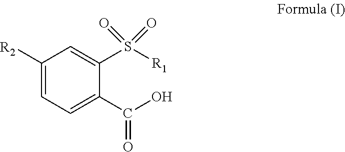

which are useful as an intermediate in the preparation of herbicidally active compounds.

### BACKGROUND OF THE INVENTION

2-alkylsulfonyl substituted benzoic acid compounds are useful as an intermediate in the preparation of pesticidal compounds such as pyrasulfotole, isoxaflutole and the like.

EP 0,527,036 patent discloses a method of preparation of 2-methylsulfonyl-4-trifluoromethyl benzoic acid by reacting 2-methylsulfenyl-4-trifluoromethyl benzoic acid with hydrogen peroxide and acetic anhydride in acetic acid. However, it does not disclose the preparation of 2-alkylsulfonyl substituted benzoic acid derivatives from 2-alkylthio substituted benzonitrile derivatives.

CN 105,646,356 patent discloses a method of preparation of 2-methylsulfonyl-4-trifluoromethyl benzoic acid by reacting methyl 2-(methylthio)-4-(trifluoromethyl)benzoate with acetic acid and hydrogen peroxide and followed by an alkaline hydrolysis. However, it does not disclose the preparation of 2-alkylsulfonyl substituted benzoic acid derivatives from 2-alkylthio substituted benzonitrile derivatives.

CN 112,010,793 patent discloses a method of preparation of 2-methylsulfonyl-4-trifluoromethylbenzoic acid by reacting with 2-methylthio-4-trifluoromethyl benzonitrile with hydrogen peroxide in presence of a metal catalyst followed by alkaline hydrolysis. However, this process requires the use of a metal catalyst and takes a longer time.

The processes described in the prior art have flaws such as long reaction time and use of metal catalyst that make the process uneconomical; and solvents are hardly recovered. Thus, there is still a need for a process that obviates the shortcomings associated with the known processes.

Therefore, there is a need to develop an effective process for the preparation of 2-alkylsulfonyl substituted benzoic acid derivatives of formula (I) from 2-alkylthio substituted benzonitrile compound of formula (II) with high yield, and selectivity is therefore desirable.

### SUMMARY OF THE INVENTION

The present invention provides a process for the preparation of a compound of formula (I):

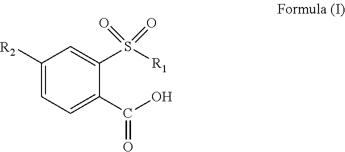

wherein
R₁ represents C₁₋₆ alkyl, C₁₋₆ haloalkyl, phenyl or phenyl substituted with carboxylic acid;
R₂ represents hydrogen, C₁₋₆ alkyl, C₁₋₆ haloalkyl, C₁₋₆ haloalkoxy, phenyl or phenyl substituted with halogen, carboxylic acid;
which comprises
   a) reacting a compound of formula (II):

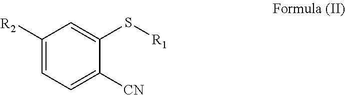

wherein R₁, and R₂ are as hereinbefore defined;
with an oxidizing reagent in the presence of an aqueous acid in a suitable solvent system to form a compound of formula (III) and the reaction is processed in the absence of a metal catalyst;

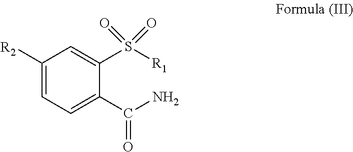

wherein R₁, and R₂ are as hereinbefore defined;
b) reacting the compound of formula (III) with an alkali hydroxide;
c) mixing the reaction mixture from step (b) with a strong acid system to obtain the compound of formula (I).

In an aspect, the present invention provides the process wherein in step (c) the strong acid system comprises the aqueous acid from step (a).

In another aspect, the present invention provides the strong acid system comprises of at least one acid stronger than compound of formula (I).

In an aspect, the present invention provides that R₁ is C₁₋₆ alkyl and R₂ is C₁₋₆ haloalkyl.

In another aspect, the present invention provides that R₁ is methyl and R₂ is trifluoromethyl.

In a further aspect, the present invention provides the oxidizing agent is hydrogen peroxide and the molar ratio of hydrogen peroxide and the compound of formula (II) is about 2:1 to 5:1, preferably about 2.5:1.

In yet another aspect, the present invention provides that the acid is selected from the group comprising sulphuric acid, phosphoric acid, nitric acid, strong carboxylic acid, or a mixture thereof and the molar ratio of acid and the compound of formula (II) is about 10:1 to 0.8:1, preferably about 1:1.

In an aspect, the present invention provides the solvent system is a mixture of an organic solvent and an aqueous solvent, wherein the organic solvent is selected from chlorobenzene, toluene, xylene, dichlorobenzene, or any other aromatic solvent inert to the reaction conditions or the mixture thereof.

In another aspect, the reaction of step (a) is carried at a temperature of about 70° C. to 110° C. and preferably 70° C. to 80° C. and the reaction mixture is cooled to a temperature of about 30° C. to 40° C. and filtered, wherein the filtrate comprises of an organic layer, and an aqueous acidic layer.

In a further aspect, the alkali hydroxide is selected from the group comprising sodium hydroxide, potassium hydroxide or calcium hydroxide, at a concentration of about 15% to 50%, preferably 45% to 50%. The alkali hydroxide and compound of formula (II) is present in a molar ratio of about 5:1 to 1:1, preferably about 1.5:1.

In yet another aspect, the present invention provides a process for the preparation of Pyrasulfotole comprising preparation of compound of formula (I) as mentioned above and further converting to Pyrasulfotole.

In an aspect, the present invention provides a process for the preparation of Isoxaflutole comprising preparation of compound of formula (I) as mentioned above and further converting to Isoxaflutole.

In a preferred aspect, the present invention provides a process for the preparation of 2-methylsulfonyl-4-trifluoromethylbenzoic acid comprises:
a) reacting 2-methylthio-4-trifluoromethyl benzonitrile with an oxidizing reagent, in the presence of an aqueous acid in a suitable solvent system, and the reaction is processed in the absence of a metal catalyst;
b) the product from step (a) reacted with alkali hydroxide and over acidification to obtain 2-methylsulfonyl-4-trifluoromethyl benzoic acid.

### DETAILED DESCRIPTION OF THE INVENTION

For the sake of clarity, specific terminology is resorted to in describing the embodiments of the invention. However, it is not intended that the invention be limited to the specific terms so selected, and it is to be understood that each specific term includes all technical equivalents that operate in a similar manner to accomplish a similar purpose.

It will be understood that the terminology used herein is for the purpose of describing embodiments only and is not intended to be limiting. As used in this specification, the singular forms “a”, “an” and “the” include plural referents unless the context clearly dictates otherwise. Thus, for example, the reference to “a compound” includes one or more of such compounds.

Unless defined otherwise, all technical and scientific terms used herein have the same meaning as commonly understood by one ordinarily skilled in the art to which the invention pertains. Although other methods and materials similar to or equivalent to those described herein can be used in the practise of the present invention, the preferred materials and methods are described herein.

As used herein, the term “or” means “and/or”. It will be further understood that the terms “comprises”, “comprising”, “includes”, “including”, or any other variation thereof, are intended to cover a non-exclusive inclusion, subject to any limitation explicitly indicated. For example, a composition or a method that comprises a list of elements is not necessarily limited to only those elements but may include other elements not expressly listed or inherent to such a composition or method.

Accordingly, unless indicated to the contrary, the numerical parameters set forth in the following specification and attached claims are approximations that may vary depending upon the desired properties sought to be obtained. At the very least, each numerical parameter should at least be construed considering the number of reported significant digits and by applying ordinary rounding techniques.

In addition, the endpoints of all ranges directed to the same component or property herein are inclusive of the endpoints, are independently combinable, and include all intermediate points and ranges.

The term “mol” refers to the quantity of a substance that reacts with an arbitrary quantity (usually one mole) of another substance in a particular chemical reaction.

As used herein, the term “alkyl” refers to a saturated aliphatic hydrocarbon group containing 1 to 6 carbon atoms. An alkyl group can be straight or branched. Examples of alkyl groups include, but not limited to, methyl, ethyl, propyl, isopropyl, butyl, isobutyl, sec-butyl, tert-butyl, n-pentyl, n-hexyl and the like.

As used herein, the term “haloalkyl” refers to an alkyl group having one or more halogen substituents (up to perhaloalkyl, i.e., every hydrogen atom of the alkyl group has been replaced by a halogen atom). A haloalkyl group can be straight or branched. For example, the term “C₁-6 haloalkyl” refers to a C₁₋₆ alkyl groups having one or more halogen substituents (up to perhaloalkyl, i.e., every hydrogen atom of the alkyl group has been replaced by a halogen atom). Examples of haloalkyl groups include, but not limited to, CF₃, C₂F₅, CHF₂, CH₂F, CH₂CF₃, CH₂Cl and the like.

As used here, the term “haloalkoxy” refers to an —O-haloalkyl group. A haloalkoxy group can be straight or branched. For example, the term “C₁₋₆ haloalkoxy” refers to an —O—(C₁₋₆ haloalkyl) group. Examples of haloalkoxy groups include, but not limited to, —OCF₃ or —OCHF₂ and the like.

As used herein, the term “carboxylic acid” refers to —C(O)—OH group having from 1 to 6 carbon atoms. A carboxylic acid group can be a straight or branched chains carboxylic acid group. Examples of carboxylic acid groups include formic acid, acetic acid, and the like.

As used herein, the term “phenyl substituted with carboxylic acid” refers to a phenyl group substituted with a carboxylic acid group. Examples include phenylacetic acid and the like.

As used herein, the term “phenyl substituted with halogen” refers to a phenyl group substituted with a halogen atom. Examples include —C₆H₄F, —C₆H₄Cl and the like.

As used herein, the term “oxidizing reagent” refers to a reagent whose oxidation potential is high enough to effect the desired reaction without significantly effecting any undesired reactions. Suitable oxidants include hydrogen peroxide and the like.

As used herein, the term “catalyst” refers to a substance that causes a change in the rate of a reaction without itself being consumed in the reaction. In the present invention it is especially applicable to catalyst suitable for the conversion of 2-alkylthio substituted benzonitrile compound of formula (II) to 2-alkylsulfonyl substituted benzoic acid compounds of formula (I). The catalyst will be referred to as metal catalyst.

As used herein, the term “suitable solvent system” refers to any solvent, and mixture of solvents, inert to the ongoing reaction that sufficiently solubilizes the reactants to afford a medium within which to effect the desired reaction.

As used herein, the term “strong acid system” refers to an acid system that comprises of at least one acid stronger than compound of formula (I) such as sulphuric acid, phosphoric acid, nitric acid, strong carboxylic acid, and mixtures thereof and the like.

As used herein the term “about” refers to and includes the values shown and the range before and after those values. In certain embodiments, the term “about” refers to ±10%, ±5%, or ±1% of the values shown.

As used herein the term, “telescopic process” refers to a chemical process that involves a process carried out in a telescopic manner without isolation of intermediates produced during the synthesis.

2-alkylsulfonyl substituted benzoic acid compound is useful as an intermediate in the preparation of agrochemical compounds. For example, it is used as an intermediate in the preparation of herbicidal actives pyrasulfotole, isoxaflutole and the like.

The present invention provides a process for the preparation of a compound of formula (I)

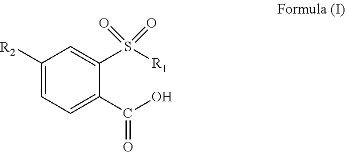

wherein
R₁ represents C₁₋₆ alkyl, C₁₋₆ haloalkyl, phenyl or phenyl substituted with carboxylic acid;
R₂ represents hydrogen, C₁₋₆ alkyl, C₁₋₆ haloalkyl, C₁₋₆ haloalkoxy, phenyl or phenyl substituted with halogen, carboxylic acid;
which comprises
   a) reacting a compound of formula (II):

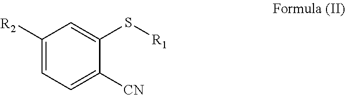

wherein R₁, and R₂ are as hereinbefore defined;
with an oxidizing reagent and in the presence of an aqueous acid in a suitable solvent system to form a compound of formula (III) and the reaction is processed in the absence of a metal catalyst;

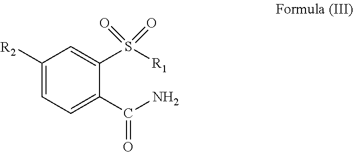

wherein R₁, and R₂ are as hereinbefore defined;
b) reacting the compound of formula (III) with an alkali hydroxide;
c) mixing the reaction mixture from step (b) with a strong acid system to obtain the compound of formula (I).

In an embodiment, the present invention provides the process wherein in step (c) the strong acid system comprises the aqueous acid from step (a).

In another embodiment, the present invention provides the strong acid system comprises of at least one acid stronger than compound of formula (I).

In an embodiment, the present invention provides that R₁ is C₁₋₆ alkyl and R₂ is C₁₋₆ haloalkyl. More preferably, R₁ is methyl and R₂ is trifluoromethyl.

The foregoing reaction is carried out with an oxidizing reagent and aqueous acid in the presence of a suitable solvent system to form a compound of formula (III).

In an embodiment, the oxidizing reagent used in the oxidation reaction may be a common oxidizing reagent, known to a person skilled in the art.

In an embodiment of the present invention, hydrogen peroxide is used as an oxidizing reagent. Additionally, to ensure the oxidation effect and assist the entire oxidation reaction, the hydrogen peroxide is added dropwise within 2 to 8 hours.

In an embodiment, the hydrogen peroxide and the compound of formula (II) is present in a molar ratio of about 2:1 to 5:1. In a preferred embodiment, the hydrogen peroxide and the compound of formula (II) is present in a molar ratio of about 2.5:1.

The acid is selected from the group comprising sulphuric acid, phosphoric acid, nitric acid, strong carboxylic acid and mixtures thereof. The strong carboxylic acid is selected from a group comprising of acid stronger than compound of formula (I) for example trilfluoroacetic acid or trichloroacetic acid and the like.

In an embodiment, the acid and the compound of formula (II) is present in a molar ratio of about 10:1 to 0.8:1. In another embodiment, the acid and the compound of formula (II) is present in a molar ratio of about 3:1 to 0.8:1. In a preferred embodiment, the acid and the compound of formula (II) is present in a molar ratio of about 1:1.

The suitable solvent according to the present invention comprises an organic solvent and an aqueous solvent, wherein the organic solvent is selected from the group comprising chlorobenzene, toluene, xylene, dichlorobenzene any other aromatic solvent inert to the reaction conditions and mixtures thereof.

The oxidation reaction of step (a) process according to the present invention is generally carried out at a temperature of about 70° C. to 110° C. In another embodiment, the temperature is about 70° C. to 100° C. More preferably at a temperature of about 70° C. to 80° C.

In a further step, to finalize oxidation process the reaction mixture was heated at 70° C. to 80° C. for 6 hours to 8 hours, cooled to a temperature of about 30° C. to 40° C. and filtered. The filtrate was phase-separated. Organic layer was used on the next step and aqueous acidic layer was kept for the last neutralization step.

In the next step, the organic layer, filter cake and a solution of alkali hydroxide was mixed and heated to a temperature of about 90° C. to 100° C. for a certain time, usually for 4 hours to 6 hours.

In a further step, water and aqueous acidic layer from the oxidation step were added and cooled to 0° C. to 5° C. for a certain time, usually 3 hours. The precipitated material was filtered to obtain the compound of formula (I).

The alkali hydroxide is selected from the group comprising sodium hydroxide, potassium hydroxide or calcium hydroxide. In an embodiment, the alkali hydroxide and compound of formula (II) is present in a molar ratio of about 5:1 to 1:1. In a further embodiment, the alkali hydroxide and compound of formula (II) is present in a molar ratio of about 3:1 to 1:1. In a preferred embodiment, the alkali hydroxide and compound of formula (II) is present in a molar ratio of about 1.5:1.

In another embodiment, the concentration of the solution of alkali hydroxide is 15% to 50%. In a further embodiment, the concentration of the solution of alkali hydroxide is 35% to 50%. In yet another embodiment, the concentration of the solution of alkali hydroxide is 40% to 50%. In a preferred embodiment, the concentration of the solution of alkali hydroxide is 48%.

The process according to the present invention is generally carried out under atmospheric pressure. However, it is also possible to carry out the process according to the invention under elevated or reduced pressure. In an embodiment, the process for preparing 2-alkylsulfonyl benzoic acid of formula (I) may be conducted at a pressure from about 1 bar to about 10 bar. In another embodiment, the process may be conducted at a pressure from about 1 bar to about 5 bar. In yet another embodiment, the process for preparing 2-alkylsulfonyl benzoic acid of formula (I) may be conducted at atmospheric pressure. In a further embodiment, the process may be conducted at about 10 bar. In certain embodiments, it may be preferred that the process is conducted at pressures less than atmospheric pressure. For example, the process may be carried out at 0.7 bar, 0.75 bar, 0.8 bar, 0.9 bar or 0.95 bar.

In yet another embodiment, the present invention provides a process for the preparation of Pyrasulfotole comprising preparation of compound of formula (I) as mentioned above and further converting to Pyrasulfotole. The compound of formula (I) can be converted to Pyrasulfotole such as described in the art for example in PCT Application No. WO 2001/074785.

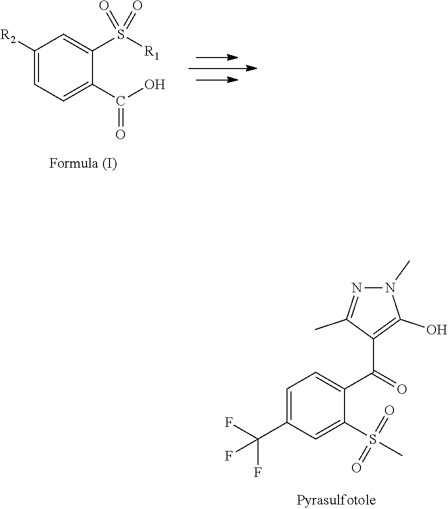

In an embodiment, the present invention provides a process for the preparation of Isoxaflutole comprising preparation of compound of formula (I) as mentioned above and further converting to Isoxaflutole.

In an embodiment, the present invention provides a process for the preparation of 2-methylsulfonyl-4-trifluoromethylbenzoic acid comprises:
a) reacting 2-methylthio-4-trifluoromethyl benzonitrile with an oxidizing reagent in the presence of an aqueous acid in a suitable solvent; and the reaction is processed in the absence of a metal catalyst;
b) the product from step (a) is reacted with alkali hydroxide and over acidification to obtain 2-methylsulfonyl-4-trifluoromethyl benzoic acid.

In another embodiment, in the above process the acid for acidification is a stronger acid than 2-methylsulfonyl-4-trifluoromethylbenzoic acid and/or an aqueous acid from step (a).

In a preferred embodiment, the present invention provides a process for the preparation of 2-methylsulfonyl-4-trifluoromethylbenzoic acid comprises:
a) reacting 2-methylthio-4-trifluoromethyl benzonitrile with hydrogen peroxide in the presence of an aqueous sulfuric acid in chlorobenzene or toluene solvent; and the reaction is processed in the absence of a metal catalyst;
b) the product from step (a) reacted with potassium hydroxide and acidification with aqueous sulphuric acid optionally the aqueous sulphuric acid from step (a) brings to 2-methylsulfonyl-4-trifluoromethyl benzoic acid.

The present process is advantageous in that it is highly efficient, providing a shorter reaction time as well as fewer effluents. It also provides high conversion and selectivity with a higher yield of the end product.

Telescopic process for preparing a 2-alkylsulfonyl substituted benzoic acid derivatives of formula (I):

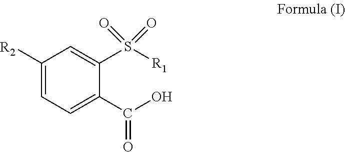

wherein
R₁ represents C₁₋₆ alkyl, C₁₋₆ haloalkyl, phenyl or phenyl substituted with carboxylic acid;
R₂ represents hydrogen, C₁₋₆ alkyl, C₁₋₆ haloalkyl, C₁₋₆ haloalkoxy, phenyl or phenyl substituted with halogen;
which comprises
a) reacting a compound of formula (II):

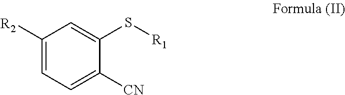

wherein R₁, and R₂ are as hereinbefore defined;
with an oxidizing reagent in the presence of an aqueous acid in a suitable solvent system to form a compound of formula (III) and the reaction is processed in the absence of a metal catalyst;

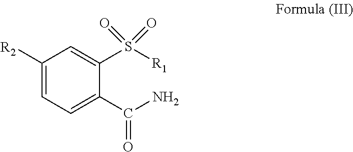

wherein R₁, and R₂ are as hereinbefore defined;
b) reacting the compound of formula (III) with an alkali hydroxide;
c) mixing the reaction mixture from step (b) with a strong acid system to obtain the compound of formula (I).

In an embodiment, the present invention provides the process wherein in step (c) the strong acid system comprises the aqueous acid from step (a).

In another embodiment, the present invention provides the strong acid system comprises of at least one acid stronger than compound of formula (I).

In a preferred embodiment, R₁ is methyl and R₂ is trifluoromethyl.

In an embodiment, the oxidizing reagent in the telescopic process is hydrogen peroxide and the molar ratio of hydrogen peroxide and the compound of formula (II) is about 5:1 to 2:1, preferably about 2.5:1.

In another embodiment, the acid in the telescopic process is sulphuric acid; and the molar ratio of sulphuric acid and the compound of formula (II) is about 10:1 to 0.8:1, preferably about 1:1.

In a further embodiment, the solvent used in the telescopic process comprises of an organic solvent and an aqueous solvent, and the organic solvent is selected from the group comprising chlorobenzene, toluene, xylene, dichlorobenzene or any other aromatic solvent inert to the reaction conditions and mixtures thereof.

In yet another embodiment, the alkali hydroxide in the telescopic process is selected from sodium hydroxide, potassium hydroxide or calcium hydroxide and the molar ratio of alkali hydroxide and compound of formula (II) is about 5:1 to 1:1, preferably about 1.5:1. The concentration of alkali hydroxide solution is about 15% to 50%, preferably 45% to 50%.

The progress of the reaction of synthesis of compound of formula (I) can be monitored using any suitable method, which can include, for example, chromatographic methods such as, e.g., high performance liquid chromatography (HPLC), thin layer chromatography (TLC), and the like. In yet another embodiment, the compound of formula (I) can be isolated from the reaction mixture by any conventional technique well-known in the art. Such isolation techniques can be selected, without limitation, from the group consisting of extraction, crystallization, or precipitation by concentration, cooling or antisolvent addition; filtration; centrifugation, and a combination thereof, followed by drying.

In yet another embodiment, the compound of formula (I) can be optionally purified by any conventional technique well-known in the art. Such purification techniques can be selected, without limitation, from the group consisting of precipitation, crystallization, extraction, slurrying, washing in a suitable solvent, filtration through a packed-bed column, dissolution in an appropriate solvent, re-precipitation by addition of a second solvent in which the compound is insoluble, and a combination thereof.

The following examples illustrate the practice of the present invention in some of its embodiments but should not be construed as limiting the scope of the present invention. From consideration of the specification and examples, other embodiments will be apparent to one skilled in the art. It is intended that the specification, including the examples, be considered exemplary only without limiting the scope and spirit of the present invention.

An exemplary experimental procedure for producing 2-methylsulfonyl-4-trifluoromethylbenzoic acid is described as follows:

### Example 1

In a four-necked flask to 2-methylthio-4-trifluoromethylbenzonitrile (106 g, 0.49 mol) in 330 g of chlorobenzene, concentrated sulphuric acid (49 g, 0.49 mol) was added and the mixture was heated to 60° C. Aqueous 30% solution of hydrogen peroxide (138.9 g, 1.23 mol) was dropped into the mixture keeping temperature below 80° C. within 3 h. The reaction mixture was heated at 70° C. to 80° C. for 8 h and cooled to 30° C. to 40° C. Precipitated compound was filtered. The resulting filtrate was phase-separated, and the lower sulphuric acid phase (177.9 g) was extracted with chlorobenzene (54.6 g). The combined organic phase (321.6 g) was directly used in the next step and the collected aqueous sulphuric acid (174.0 g) for acidification in the next step.

The organic phase (321.6 g) and the filter cake (167.0 g) were mixed with an aqueous solution of 48% of potassium hydroxide (87.7 g, 0.75 mol) in a four-necked flask and the mixture was heated to 90° C. to 95° C. The mixture was stirred at this temperature for 5 h. Water (813.2 g) and aqueous sulphuric acid phase from previous step were added to the mixture and stirred for 1 h at 90° C. to 100° C. The reaction mixture was cooled to 0° C. to 5° C. within 3 h and filtered to collect 2-methylsulfonyl-4-trifluoromethylbenzoic acid. Solid was rinsed with water (54.6 g) and cooled chlorobenzene (54.6 g). The filter cake was dried at 60° C. to 65° C. to obtain an off-white solid (118.4 g). Yield of 2-methylsulfonyl-4-trifluoromethylbenzoic acid 89%.

### Example 2

In a four-necked flask to 2-methylthio-4-trifluoromethylbenzonitrile (106 g, 0.49 mol) in 300 g of toluene, concentrated sulphuric acid (49 g, 0.49 mol) was added and the mixture was heated to 60° C. Aqueous 30% solution of hydrogen peroxide (138.9 g, 1.23 mol) was dropped into the mixture keeping temperature below 80° C. within 3 h. The reaction mixture was heated at 70° C. to 80° C. for 8 h and cooled to 30° C. to 40° C. Precipitated compound was filtered. The resulting filtrate was phase-separated, and the lower sulphuric acid phase was extracted with toluene. The combined organic phase (about 320 g) was directly used in the next step and the collected aqueous sulphuric acid (about 170 g) for acidification in the next step.

The organic phase (about 320 g) and the filter cake (164.0 g) were mixed with an aqueous solution of 18% of potassium hydroxide (235 g, 0.75 mol) in a four-necked flask and the mixture was heated to 90° C. to 95° C. The mixture was stirred at this temperature for 10 h. Water (700 g) and aqueous sulphuric acid phase from previous step were added to the mixture and stirred for 1 h at 90° C. to 95° C. The reaction mixture was cooled to 0° C. to 5° C. within 3 h and filtered to collect 2-methylsulfonyl-4-trifluoromethylbenzoic acid. Solid was rinsed with water (50 g) and cooled toluene (50 g). The filter cake was dried at 60° C. to 65° C. to obtain an off-white solid (113 g). Yield of 2-methylsulfonyl-4-trifluoromethylbenzoic acid 85%.

We claim:

1. A process for the preparation of a compound of formula (I): 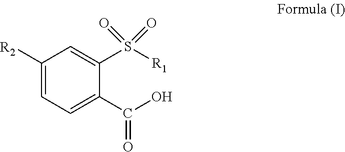
wherein
R₁ represents C₁₋₆ alkyl, C₁₋₆ haloalkyl, phenyl or phenyl substituted with carboxylic acid;
R₂ represents hydrogen, C₁₋₆ alkyl, C₁₋₆ haloalkyl, C₁₋₆ haloalkoxy, phenyl or phenyl substituted with halogen, carboxylic acid;
which comprises
a) reacting a compound of formula (II): 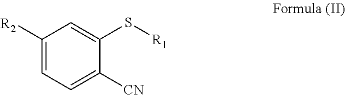
wherein R₁, and R₂ are as hereinbefore defined;
with an oxidizing reagent in the presence of an aqueous acid in a suitable solvent system to form a compound of formula (III) and the reaction is processed in the absence of a metal catalyst; 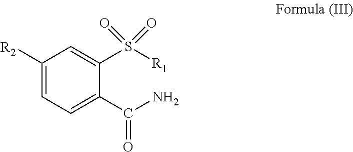
wherein R₁, and R₂ are as hereinbefore defined;
b) reacting the compound of formula (III) with an alkali hydroxide;
c) mixing the reaction mixture from step (b) with a strong acid system to obtain the compound of formula (I).

2. The process as claimed in claim 1, wherein in step (c) the strong acid system comprises the aqueous acid from step (a).

3. The process as claimed in any of claims 1-2, wherein the strong acid system comprises of at least one acid stronger than compound of formula (I).

4. The process as claimed in claim 1, wherein R₁ is C₁₋₆ alkyl and R₂ is C₁₋₆ haloalkyl.

5. The process as claimed in claim 4, wherein R₁ is methyl and R₂ is trifluoromethyl.

6. The process as claimed in claim 1, wherein the oxidizing reagent is hydrogen peroxide.

7. The process as claimed in claim 6, wherein the hydrogen peroxide and the compound of formula (II) is present in a molar ratio of about 5:1 to 2:1.

8. The process as claimed in claim 6, wherein the hydrogen peroxide is added into the reaction mixture within 2 to 8 hours in a dropping mode.

9. The process as claimed in claim 1, wherein the acid is selected from the group comprising of sulphuric acid, phosphoric acid, nitric acid, strong carboxylic acid, and mixtures thereof.

10. The process as claimed in claim 1, wherein the acid and the compound of formula (II) is present in a molar ratio of about 10:1 to 0.8:1.

11. The process as claimed in claim 1, wherein the solvent system comprises of an organic solvent and an aqueous solvent.

12. The process as claimed in claim 11, wherein the organic solvent is selected from chlorobenzene, toluene, xylene, dichlorobenzene or any other aromatic solvent inert to the reaction conditions and mixtures thereof.

13. The process as claimed in claim 1, wherein the reaction of step (a) is carried at a temperature of about 70° C. to 110° C.

14. The process as claimed in claim 1, wherein the reaction mixture of step (a) is cooled to a temperature of about 30° C. to 40° C. and filtered.

15. The process as claimed in claim 14, wherein the filtrate from step (a) comprises of an organic layer, and an aqueous acidic layer.

16. The process as claimed in claim 1, wherein the alkali hydroxide is selected from the group comprising of sodium hydroxide, potassium hydroxide or calcium hydroxide.

17. The process as claimed in claim 1, wherein the alkali hydroxide and compound of formula (II) is present in a molar ratio of about 5:1 to 1:1.

18. The process as claimed in claim 1, wherein the concentration of alkali hydroxide solution is about 15% to 50%.

19. The process as claimed in claim 15, wherein the organic layer, a filter cake from step (a) and the alkali hydroxide was heated at a temperature of about 90° C. to 100° C.

20. The process as claimed in claim 1, wherein the reaction mixture from step (c) is cooled to a temperature of about 0° C. to 5° C. and filtered to obtain the compound of formula (I).

21. A process for the preparation of Pyrasulfotole comprising preparation of compound of formula (I) as claimed in claim 1 and further converting to Pyrasulfotole.

22. A process for the preparation of 2-methylsulfonyl-4-trifluoromethylbenzoic acid comprises:
a) reacting 2-methylthio-4-trifluoromethylbenzonitrile with an oxidizing reagent, in the presence of an aqueous acid in a suitable solvent system, and the reaction is processed in the absence of a metal catalyst;
b) the product from step (a) is reacted with alkali hydroxide and over acidification to obtain 2-methylsulfonyl-4-trifluoromethylbenzoic acid.

23. The process as claimed in claim 22, wherein the oxidizing reagent is hydrogen peroxide and the molar of hydrogen ratio peroxide and 2-methylthio-4-trifluoromethylbenzonitrile is about 5:1 to 2:1.

24. The process as claimed in claim 22, wherein the acid is sulphuric acid, phosphoric acid, nitric acid, strong carboxylic acid, and mixtures thereof; and the molar ratio of acid and 2-methylthio-4-trifluoromethylbenzonitrile is about 10:1 to 0.8:1.

25. The process as claimed in claim 22, wherein the solvent comprises of an organic solvent and an aqueous solvent, and the organic solvent is selected from the group comprising chlorobenzene, toluene, xylene, dichlorobenzene or any other aromatic solvent inert to the reaction conditions and mixtures thereof.

26. The process as claimed in claim 22, wherein the alkali hydroxide is selected from sodium hydroxide, potassium hydroxide or calcium hydroxide and the molar ratio of alkali hydroxide and 2-methylthio-4-trifluoromethylbenzonitrile is about 5:1 to 1:1.

27. The process as claimed in claim 22, wherein the concentration of alkali hydroxide solution is about 15% to 50%.
